(19) United States
(12) Patent Application Publication
(10) Pub. No.: US 2016/0296577 A1
(43) Pub. Date: Oct. 13, 2016

(54) HERBAL COMPOSITION, PROCESS FOR ITS PREPARATION AND USE THEREOF

(71) Applicant: PIRAMAL ENTERPRISES LIMITED, Mumbai (IN)

(72) Inventors: Swati Ajay PIRAMAL, Mumbai, Maharashtra (IN); Ashish Chandrakant SUTHAR, Mumbai, Maharashtra (IN); Rahul Ramesh SHELAR, Mumbai, Maharashtra (IN)

(73) Assignee: PIRAMAL ENTERPRISES LIMITED, Mumbai (IN)

(21) Appl. No.: 15/031,889

(22) PCT Filed: Oct. 28, 2014
(86) PCT No.: PCT/IB2014/065649

§ 371 Date: Apr. 25, 2016

Related U.S. Application Data
(60) Provisional application No. 61/896,321, filed on Oct. 28, 2013.

(51) Int. Cl. A61K 36/42; A61K 31/7076; A61K 31/7072

(52) U.S. Cl. CPC A61K 36/42; A61K 31/7072; A61K 31/7076; A61K 2236/31; A61K 2236/13; A61K 2236/15

(57) ABSTRACT

The present invention relates to a standardised extract of Momordica Charantia containing one or more nitrogen containing heterocyclic compounds as the bioactive markers and a process for the preparation thereof. Tile present invention also relates to a composition comprising the standardised extract of Momordica Charantia containing one or more nitrogen containing heterocyclic compounds as the bioactive markers. The present invention also relates to use of the standardised extract of Momordica Charantia or the composition containing the said standardised extract for the treatment of metabolic disorders such as diabetes.

### TECHNICAL FIELD

The present invention relates to an extract of Momordica Charantia that contains one or more nitrogen containing heterocyclic compounds (as described herein) as bioactive markers; a process for preparation of the said extract; a composition containing the said extract and use thereof for the treatment of metabolic disorders.

### BACKGROUND OF THE INVENTION

Metabolic disorders occur when the body is unable to properly metabolise carbohydrates, lipids, proteins, or nucleic acids. Most metabolic disorders are caused by genetic mutations that result in missing or dysfunctional enzymes that are needed for the cell to perform metabolic processes. Examples of metabolic disorders include obesity, excessive body fat, hyperlipidemia, hyperlipoproteinemia, hyperglycemia, hypercholesterolemia, hyperinsulinemia, insulin resistance, glucose intolerance, and diabetes mellitus (diabetes), particularly type 2 diabetes.

Among the metabolic disorders, diabetes mellitus is the most prevalent, and is considered to be one of the five leading causes of death in the world. There are more than 150 million people suffering from diabetes worldwide and it is expected that this figure will be over 366 million by 2030. It is a syndrome of metabolism, usually due to combination of hereditary and environmental causes, leading to abnormal increase in blood sugar levels (hyperglycemia).

There are two types of diabetes: Insulin dependent Diabetes Mellitus (IDDM or Type 1 diabetes) and Non-insulin dependent Diabetes Mellitus (NIDDM or Type 2 diabetes). Type 1 diabetes is an autoimmune disease frequently occurring in children and young adults. Type 2 diabetes, the most common type of diabetes, results from the body's inability to produce insulin in sufficient amount or to properly use the insulin that it produces. Defects in insulin secretion and insulin resistance may be considered as the main causes of Type 2 diabetes. Diabetes mellitus, including the Type 2 diabetes, is known to be associated with secondary complications such as cardiovascular disease, peripheral vascular disease, stroke, diabetic neuropathy, diabetic nephropathy, and diabetic retinopathy.

Obesity is another prevalent health problem affecting all age groups. Among adults, 300 millions are suffering from obesity, and this figure tends to increase quickly, resulting in a rapid increase in obesity-related diseases, such as type 2 diabetes, cardiac diseases, stroke and hypertension. The major reasons resulting in overweight and obesity are attributed to high fatty and high calorie diet, lack of exercise and the accelerating urbanization. Considerable efforts have been taken to develop anti-obesity drugs, however, there still does not exist an ideal anti-obesity drug that would produce sustained weight loss with minimal side effects. Further, very few drugs have been approved for the treatment of obesity from among the investigational drugs. Moreover, from among the approved anti-obesity drugs namely orlistat, a specific inhibitor of gastrointestinal tract lipases, and sibutramine, a monoamine reuptake inhibitor, the latter, i.e., sibutramine has been discontinued in a few countries including India and the US owing to its adverse cardiovascular effects.

The well known drugs for diabetes like sulfonylureas, glinides, glucagon-like peptide 1 (GLP-1) receptor agonists, metformin, thiazolidinediones and α-glucosidase inhibitors, generally target only insulin resistance or β-cell dysfunction by increasing insulin secretion or tissue sensitivity to insulin. In addition, substantial number of marketed drugs is associated with major drawbacks that limit the efficiency of therapy. Among others, the following problems continue to plague current therapy: 1) hypoglycemia (especially when initiating therapy; severe hypoglycaemia is known to lead to myocardial infarction and to the development of dementia); 2) rise in weight gain (a leading factor driving the epidemic of diabetes); 3) increase in insulin resistance; and 4) β-cell destruction. This clearly establishes a requirement for new therapies or further improvement in current therapies to overcome these drawbacks.

Thus, in view of the fact that currently available treatment options for diabetes are associated with certain drawbacks, there is a continuing need for effective therapy for Type 2 diabetes; its associated disorders and complications associated with it.

Plant based medicines have been used for the treatment of several diseases, including diabetes considering that plants provide for alternative treatment option. Momordica Charantia, a flowering vine in the family Cucurbitaceae, is also known as bitter melon, bitter gourd, karela, balsam pear, and it has been very popular plant used for the treatment of diabetes (Int J Diabetes & Metabolism 2003. 11: pages 46-55). Besides, it has been widely used as a medicinal remedy for dispelling “heat”, detoxicating, improving acuity of vision, invigorating stomach, relieving thirst and as a helminthicide.

The hypoglycemic activity of Momordica Charantia, both in human and animal models, has been reported. In fact, extracts derived from Momordica Charantia are known to be commercially available for the treatment of diabetes. These extracts either alone or in combination with other therapeutic agents, are used for the treatment of diabetes. Despite the available extracts of Momordica Charantia for use in the treatment of diabetes, there is a need to provide an improved composition containing a standardised extract of Momordica Charantia that would effectively treat diabetes and its associated disorders including the complications associated with it.

The extraction processes of the active constituents from various parts of Momordica Charantia have been reported in prior art documents. The extraction processes and the formulation methods of active constituents from Momordica Charantia have been disclosed in Patent Documents: 181887, JP2006314273, GB1435664, WO2013123912, JP2005126370, IN156263, U.S. Pat. No. 5,098,710, CN1180545, CN101637491, U.S. Pat. No. 6,852,695, U.S. Pat. No. 6,831,162, IN191582, CN1253734, CN1303698, IN188858, IN826/DEL/2000, IN768/MUM/2001, CN1418890, CN1562340, CN1858223, CN1709900, CN1872134, JP2008120701, TW200927139, CN101366806, CN101461514, CN101485429 and CN 101597389.

While treatment of diabetes involving use of the commercial extracts derived from Momordica Charantia is known, certain drawbacks associated with the extracts have been identified, especially in terms of the extracts having limited efficacy, the requirement of prolonged intake or longer duration of therapy, the presence of neurotoxic elements present in the extracts. Vicine is a one type of neurotoxin (International Journal of Toxicology 2002, 21: pages 201-209), which is present in commercially available extracts derived from Momordica Charantia. Another drawback associated with the commercially available extracts of Momordica Charantia, is that the extracts are relatively low in natural Vitamin C and natural fibre contents; which are essential nutrients for a diabetic patient since they serve as antioxidant and carbohydrate tolerance improver.

Thus, there is a need to provide improved compositions containing an extract, particularly, a standardised extract of Momordica Charantia having improved efficacy and safety that would effectively treat diabetes and the secondary complications associated with it.

### SUMMARY OF THE INVENTION

In one aspect, the present invention relates to an extract of Momordica Charantia wherein the said extract contains one or more nitrogen containing heterocyclic compounds (as described herein) as the bioactive markers.

In another aspect, the present invention relates to a standardized extract of Momordica Charantia containing one or more nitrogen containing heterocyclic compounds selected from the group consisting of uridine, adenine and 2-hydroxy adenosine; such that the said extract is standardised in relation to one or more of the said nitrogen containing heterocyclic compounds as the bioactive markers.

In another aspect, the present invention relates to an extract of Momordica Charantia containing one or more nitrogen containing heterocyclic compounds selected from the group consisting of uridine, adenine and 2-hydroxy adenosine as the bioactive markers; wherein the said extract is enriched with natural vitamin C and fibres, and wherein the said extract is substantially free from vicine.

In another aspect, the present invention relates to a process for preparation of the extract of Momordica Charantia containing one or more nitrogen containing heterocyclic compounds selected from the group consisting of uridine, adenine and 2-hydroxy adenosine as bioactive markers.

In another further aspect, the present invention relates to a composition comprising a therapeutically effective amount of an extract of Momordica Charantia containing one or more nitrogen containing heterocyclic compounds selected from the group consisting of uridine, adenine and 2-hydroxy adenosine as the bioactive markers, either alone, or along with at least one pharmaceutically acceptable excipient.

In yet another aspect, the present invention relates to a method of treating a metabolic disorder comprising administering to a subject in need thereof a therapeutically effective amount of the extract of Momordica Charantia (as described herein).

In yet another aspect, the present invention relates to a method of treating a metabolic disorder comprising administering to a subject in need thereof a therapeutically effective amount of the composition comprising the extract of Momordica Charantia (as described herein).

In another aspect, the present invention relates to a method of treating diabetes or secondary complications associated with diabetes comprising administering to a subject in need thereof a therapeutically effective amount of an extract of Momordica Charantia containing one or more nitrogen containing heterocyclic compounds selected from the group consisting of uridine, adenine and 2-hydroxy adenosine as the bioactive markers.

In another aspect, the present invention relates to a method of treating diabetes or secondary complications associated with diabetes comprising administering to a subject in need thereof a composition comprising therapeutically effective amount of an extract of Momordica Charantia containing one or more nitrogen containing heterocyclic compounds selected from the group consisting of uridine, adenine and 2-hydroxy adenosine as the bioactive markers.

According to another aspect, the present invention relates to an extract of Momordica Charantia containing one or more nitrogen containing heterocyclic compounds (as described herein) as the bioactive markers or a composition containing the said extract; for use in combination with a further therapeutically active agent for the treatment of a metabolic disorder, particularly diabetes or secondary complications associated with diabetes.

These and other aspects and advantages of the present invention will be apparent to those skilled in the art from the following description.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the description herein, may utilize the present invention to its fullest extent. The following specific embodiments are to be construed as merely illustrative, and not limitative of the remainder of the disclosure in any way whatsoever.

Unless otherwise defined, all the terms used herein, including the technical and scientific terms, have the meaning as that generally understood by one of ordinary skill in the art to which the present invention relates.

### Definitions

It should be noted that, as used in this specification and the appended claims, the singular forms “a,” “an,” and “the” include plural referents unless the content clearly dictates otherwise.

It should also be noted that the term “and” is generally employed in its sense including “and/or” unless the content clearly dictates otherwise.

As used herein, the term “nitrogen containing heterocyclic compounds” refers to a group of compounds including a nucleobase such as adenine and/or a nucleoside such as uridine and 2-hydroxy adenosine. Accordingly, “the nitrogen containing heterocyclic compounds” contained in the extract as the bioactive markers, include, but may not be limited to, adenine, uridine and 2-hydroxy adenosine.

The term “one or more” as used in reference to the nitrogen containing heterocyclic compounds (as described herein) means one to two nitrogen containing heterocyclic compounds; preferably, one to three nitrogen containing heterocyclic compounds.

The term “subject” as used herein refers to animals including, but not limited to, any mammals, in particular humans or non-human mammals. Non-human mammals include, but are not limited to, domestic animals, such as cows, pigs, horses, dogs, cats, rabbits, rats and mice, and non-domestic animals. In the context of the present invention, the term “subject” may be used interchangeably with the term “patient”. In the context of the present invention, the phrase “a subject in need thereof” means a subject (patient) in need for the treatment of a disease or disorder for which the extract of Momordica Charantia (as described herein) or the composition comprising the said extract (as described herein) can be suitably used.

The term “treatment”, “treat” or “treating” as used herein means alleviating, inhibiting, slowing or arresting the development, reversing and/or relieving the conditions (e.g. secondary complications associated with diabetes), diseases, disorders (e.g. metabolic disorders such as diabetes) or syndromes to which such term is associated with. Treatment also includes preventing development of or alleviating to some extent, one or more of the symptoms of the disease, disorder or condition being treated.

The term “therapeutically effective amount” or “effective amount” as used herein means an amount of the therapeutically active compound (e.g. the extract of Momordica Charantia as described herein) sufficient to effect beneficial or desired results for treating a condition, disease, disorder, state or syndrome. In the context of the present invention, the condition, disease or disorder refers to metabolic disorders such as diabetes or secondary complications associated with diabetes. An effective amount can be administered in one or more administrations. An effective amount is typically sufficient to palliate, ameliorate, stabilize, reverse, slow or delay the progression of the disease state.

Generally, the term “metabolic disorders” refers to the disorders or defects that occur when the body is unable to properly metabolise carbohydrates, lipids, proteins, or nucleic acids. In the context of the present invention, the metabolic disorder is selected from, but is not limited to, insulin resistance, hyperglycemia, diabetes (type 1 or type 2 diabetes), secondary complications associated with diabetes, obesity, glucose intolerance, hypercholesterolemia, dyslipidemia, hyperinsulinemia, atherosclerotic disease, polycystic ovary syndrome, coronary artery disease, metabolic syndrome, hypertension, or a related disorder associated with abnormal plasma lipoprotein, triglycerides or a disorder related to glucose levels such as pancreatic beta cell regeneration.

The term “pharmaceutically acceptable” as used herein means the carrier, diluent, and/or excipients used in the composition must be compatible with the other ingredients of the formulation, and not deleterious to the recipient thereof.

The term “pharmaceutically acceptable excipient” as used herein means a non-toxic, inert solid, semi-solid, diluent, encapsulating material or formulation auxiliary of any type. Some examples of materials which can serve as pharmaceutically acceptable excipient are sugars such as lactose, glucose, and sucrose; starches such as corn starch and potato starch; cellulose and its derivatives such as sodium carboxymethyl cellulose, ethyl cellulose and cellulose acetate; malt; gelatin: as well as other non-toxic compatible lubricants such as sodium lauryl sulfate and magnesium stearate, as well as coloring agents, releasing agents, coating agents, sweetening, flavoring and perfuming agents; preservatives and antioxidants can also be used in the composition, according to the judgment of the formulator.

The term “either alone” may indicate that the composition contains only the extract of Momordica Charantia, particularly, the standardised extract of Momordica Charantia, as described herein, without any pharmaceutically acceptable excipient added therein. It should be noted that the term “composition” should be construed in a broad sense and includes any composition which is intended for the purpose of achieving a therapeutic effect whether sold as a pharmaceutical product, for example carrying a label as to the intended indication, whether sold over the counter, or whether sold as a phytopharmaceutical.

The term “standardized extract”, as used herein, refers to an extract of a plant e.g. Momordica Charantia or a part of the plant, which contains one or more bioactive markers (bioactive substances) in appropriate concentration. For instance, the extract may be standardized to contain about 0.10% to about 5.00% by weight of total nitrogen containing heterocyclic compounds selected from the group consisting of uridine, adenine and 2-hydroxy adenosine as the bioactive markers. The total amount in which the nitrogen containing heterocyclic compounds are contained in the extract of Momordica Charantia can be more than 5% by weight of the said nitrogen containing heterocyclic compounds. In the context of the present invention, use of the term “extract of Momordica charantia” or “extract” may refer to “standardized extract of Momordica charantia”.

As used herein, the term “about” means approximately and in the context of numerical values the term “about” can be construed to estimate a value that is ±10%, preferably, ±5% of the value or range recited.

The term “bioactive markers” is used herein to define a characteristic (or a phytochemical profile) of an active compound/compounds which is correlated with an acceptable degree of pharmaceutical or therapeutic activity. A “bioactive marker”, which is the active compound, may be isolated from the extract obtained from Momordica charantia by preparative HPLC or any other method known in the art. The term “bioactive ingredients” may be used exchangeable with the term “bioactive markers” and have the same meaning as the term “bioactive markers”.

### Embodiments

In an embodiment, the present invention relates to an extract of Momordica Charantia containing one or more nitrogen containing heterocyclic compounds selected from the group consisting of uridine, adenine and 2-hydroxy adenosine as the bioactive markers.

In an embodiment, the present invention relates to an extract of Momordica Charantia containing one to three nitrogen containing heterocyclic compounds selected from the group consisting of uridine, adenine and 2-hydroxy adenosine as the bioactive markers.

In another embodiment, the present invention relates to an extract of Momordica Charantia containing uridine, and 2-hydroxy adenosine as the bioactive markers.

In another embodiment, the present invention relates to an extract of Momordica Charantia containing uridine and adenine as the bioactive markers.

In yet another embodiment, the present invention relates to an extract of Momordica Charantia containing uridine and 2-hydroxy adenosine as the bioactive markers.

In another embodiment, the present invention relates to an extract of Momordica Charantia containing adenine and 2-hydroxy adenosine as the bioactive markers.

In another embodiment, the present invention relates to an extract of Momordica Charantia containing uridine, adenine and 2-hydroxy adenosine as the bioactive markers.

According to yet another embodiment of the present invention, the extract of Momordica Charantia recited in one or more embodiments stated above is enriched with natural vitamin C and fibres.

In an embodiment, the present invention relates to an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres.

In an embodiment, the present invention relates to an extract of Momordica Charantia containing one to three nitrogen containing heterocyclic compounds selected from the group consisting of uridine, adenine and 2-hydroxy adenosine as the bioactive markers; wherein said extract is enriched with natural vitamin C and fibres.

In another embodiment, the present invention relates to an extract of Momordica Charantia containing uridine, adenine and 2-hydroxy adenosine as the bioactive markers; wherein said extract is enriched with natural vitamin C and fibres.

According to an embodiment, the extract of Momordica Charantia recited in one or more embodiments stated above is substantially free from a neurotoxic substance.

In another embodiment, the neurotoxic substance is vicine.

Thus, according to an embodiment, the present invention relates to an extract of Momordica Charantia containing one or more nitrogen containing heterocyclic compounds selected from the group consisting of uridine, adenine and 2-hydroxy adenosine as the bioactive markers; and wherein said extract is enriched with natural vitamin C and fibres: wherein the said extract is substantially free from vicine.

Thus, according to an embodiment, the present invention relates to an extract of Momordica Charantia containing one to three nitrogen containing heterocyclic compounds selected from the group consisting of uridine, adenine and 2-hydroxy adenosine as the bioactive markers; and wherein said extract is enriched with natural vitamin C and fibres; wherein the said extract is substantially free from vicine.

Thus, according to an embodiment, the present invention relates to an extract of Momordica Charantia containing uridine, adenine and 2-hydroxy adenosine as the bioactive markers; and wherein said extract is enriched with natural vitamin C and fibres; wherein the said extract is substantially free from vicine.

The term “substantially free from neurotoxic substance” or “substantially free from vicine” as used herein means an extract of Momordica Charantia recited in one or more embodiments stated above; having a weight content of a neurotoxic substance or more specifically vicine from 0% to about 0.004%; preferably below about 0.002%, and more preferably 0%. All of these percentages, unless otherwise stated, refer to percent by weight.

In another embodiment, the present invention relates to an extract of Momordica Charantia containing from about 0.1% to about 5% by total weight of the nitrogen containing heterocyclic compounds selected from the group consisting of uridine, adenine and 2-hydroxy adenosine as the bioactive markers.

In another embodiment, the present invention relates to an extract of Momordica Charantia containing from about 0.1% to about 3% by total weight of the nitrogen containing heterocyclic compounds selected from the group consisting of uridine, adenine and 2-hydroxy adenosine as the bioactive markers.

In another embodiment, the present invention relates to an extract of Momordica Charantia containing from about 0.1% to about 1% by total weight of the nitrogen containing heterocyclic compounds selected from the group consisting of uridine, adenine and 2-hydroxy adenosine as the bioactive markers.

In another embodiment, the present invention relates to an extract of Momordica Charantia containing from 0.1% to 3% of uridine, 0.1% to 3% of adenine and 0.1% to 3% of 2-hydroxy adenosine as the bioactive markers; wherein said extract is enriched with natural vitamin C and fibres.

In the context of the present invention, the extract of Momordica Charantia containing one or more nitrogen containing heterocyclic compounds selected from the group consisting of uridine, adenine and 2-hydroxy adenosine as the bioactive markers, is referred to herein as a standardised extract.

In an embodiment, the standardised extract refers to the extract of Momordica Charantia containing uridine, adenine and 2-hydroxy adenosine as the bioactive markers.

In an embodiment, the standardised extract refers to the extract of Momordica Charantia containing uridine, adenine and 2-hydroxy adenosine as the bioactive markers; wherein said extract is enriched with natural vitamin C and fibres; wherein the said extract is substantially free from vicine.

In an aspect, the present invention relates to a process for the preparation of a standardised extract of Momordica Charantia comprising the steps of:
a. preparing juice from fresh unripe green fruits of Momordica Charantia along with seeds;
b. filtering the juice as obtained in step (a) to obtain an extract with suspended particles;
c. altering pH of the juice extract as obtained in step (b) to the acidic pH range by the addition of a natural tonic;
d. allowing the juice extract as obtained in step (c) to stand,
e. neutralising the pH of the juice extract as obtained in step (d) by using a base or alkali;
f. adding excipients to the juice extract as obtained in step (e) to obtain a homogenous mixture;
g. concentrating the juice extract as obtained in step (f) to a semi solid mass under distillation;
h. drying the concentrated juice extract as obtained in step (g) to obtain dried extract of Momordica Charantia; and
i. determining the amount of bioactive markers in the extract of Momordica Charantia; wherein the bioactive markers are nitrogen containing heterocyclic compounds selected from the group consisting of uridine, adenine and 2-hydroxy adenosine.

In the process as described above, in step (i) the amount of the bioactive markers is determined by using chromatographic methods such as HPLC method.

In one embodiment, water is optionally added while preparing the juice of the fruits of Momordica Charantia as described in step (a).

In one embodiment, natural tonic used in step (c) is Amla pulp.

In another embodiment, in step (c) of the process, the acidic pH range of the juice extract is 2 to 6.

In another embodiment, in step (c) of the process, the acidic pH range of the juice extract is 4 to 4.5.

In another embodiment, in step (c) of the process, the acidic pH of the juice extract is 4.5.

In another embodiment, in step (d) of the process, the juice extract is allowed to stand for 1 minute to 2 hours.

In another embodiment, in step (d) of the process, the juice extract is allowed to stand for 1 minute to 1 hour.

In another embodiment, in step (d) of the process, the juice extract is allowed to stand for 5 to 30 minutes.

In another embodiment, in step (e) of the process, the base or alkali is selected from the group consisting of sodium hydroxide, potassium hydroxide, calcium hydroxide, sodium bicarbonate, potassium bicarbonate and calcium bicarbonate.

In another embodiment, in step (e) of the process, the alkali is sodium hydroxide.

In yet another embodiment, in step (e) of the process, sodium hydroxide is used as the alkali, in an amount ranging from 0.05% to 0.25%.

In another embodiment, in step (e) of the process, sodium hydroxide is used as the alkali in an amount of 0.15%.

In another embodiment, in step (e) of the process, the alkali is added dropwise, optionally with stirring.

In another embodiment, in step (f) of the process, the excipients are added under continuous stirring.

In another embodiment, in step (f) of the process, the excipients are microcrystalline cellulose and Aerosil.

In an embodiment, the present invention relates to a process for the preparation of a standardised extract of Momordica Charantia comprising the steps of:
(i) preparing crude juice by crushing the fresh unripe green fruits of Momordica Charantia along with seeds wherein water is added intermittently;
(ii) filtering the crude juice as obtained in step (i) to obtain clear juice extract with suspended particles;
(iii) altering the pH of the juice extract as obtained in step (ii) between 4 to 4.5 by the addition of a natural tonic;
(iv) allowing the juice extract as obtained in step (iii) to stand for 5 to 30 minutes;
(v) neutralising the pH of the juice extract as obtained in step (iv) by adding dropwise an alkali with continuous stirring;
(vi) adding excipients to the juice extract as obtained in step (v) under continuous stirring to obtain homogenous mixture;
(vii) concentrating the juice extract as obtained in step (vi) to semi solid mass under distillation and reduced pressure;
(viii) drying the concentrated juice extract as obtained in step (vii) to obtain dried extract of Momordica Charantia;
(ix) determining the amount of bioactive markers in the extract of Momordica Charantia by using a chromatographic method.

In the process as described above, in step (ix) the amount of the bioactive markers is determined by using HPLC method as the chromatographic method.

In another embodiment, the present invention relates to an extract of Momordica Charantia obtained by the process described herein.

In one aspect, the present invention relates to a composition comprising an extract of Momordica Charantia as recited in one or more embodiments stated above.

In one aspect, the present invention relates to a composition comprising an extract of Momordica Charantia containing one or more nitrogen containing heterocyclic compounds selected from the group consisting of uridine, adenine and 2-hydroxy adenosine as the bioactive markers, and wherein the said composition contains the extract either alone or along with at least one pharmaceutically acceptable excipient.

In an embodiment, the present invention relates to a composition comprising an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres, and wherein the said composition contains the extract either alone or along with at least one pharmaceutically acceptable excipient.

In an embodiment, the present invention relates to a composition comprising an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres; and is substantially free from vicine; and wherein the said composition contains the extract either alone or along with at least one pharmaceutically acceptable excipient.

In an embodiment, the present invention relates to a composition comprising an extract of Momordica Charantia containing uridine, adenine and 2-hydroxy adenosine as the bioactive markers; and wherein the said composition contains the extract either alone or along with at least one pharmaceutically acceptable excipient.

In an embodiment, the present invention relates to a composition comprising an extract of Momordica Charantia containing uridine, adenine and 2-hydroxy adenosine as the bioactive markers; wherein said extract is enriched with natural vitamin C and fibres, and wherein the said composition contains the extract either alone or along with at least one pharmaceutically acceptable excipient.

In an embodiment, the present invention relates to a composition comprising an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres; and is substantially free from vicine; and wherein the said composition contains the extract either alone or along with at least one pharmaceutically acceptable excipient.

In an embodiment, the present invention relates to a composition comprising an extract of Momordica Charantia containing uridine, adenine and 2-hydroxy adenosine as the bioactive markers; wherein said extract is enriched with natural vitamin C and fibres; and is substantially free from vicine; and wherein the said composition contains the extract either alone or along with at least one pharmaceutically acceptable excipient.

The composition of the present invention as described herein constitutes a herbal composition considering that the said composition comprises an extract of Momordica Charantia as recited in one or more embodiments stated above.

In another embodiment, the extract contained in the composition of the present invention is dried extract of Momordica Charantia.

In another embodiment, the composition of the present invention contains 0.1% to 10% by weight of the extract of Momordica Charantia as recited above in one or more embodiments of the present invention.

In another embodiment, the composition of the present invention contains 0.1% to 5% by weight of the extract of Momordica Charantia as recited above in one or more embodiments of the present invention.

In another embodiment, the composition of the present invention contains 0.1% to 3% by weight of the extract of Momordica Charantia as recited above in one or more embodiments of the present invention.

In an embodiment, the composition of the present invention is provided for oral administration.

In another embodiment, the composition of the present invention can be orally administered in a dosage form selected from, but not limited to, powder, granule, capsule, tablet, sachet, suspension, liquid, pastille, chewing gum, lozenges or pill.

The herbal composition of the present invention may be formulated for oral administration by compounding the active ingredient i.e. the extract of the plant Momordica Charantia which may be a standardized extract with the usual non-toxic pharmaceutically acceptable excipient/s for powders, pills, tablets, coated tablets, pellets, granules, capsules, solutions, emulsions, suspensions, elixirs, syrup, and any other form suitable for use. Formulations of the present invention encompass those which include talc, water, glucose, lactose, sucrose, gum acacia, gelatin, mannitol, starch paste, magnesium trisilicate, corn starch, keratin, colloidal silica, potato starch, urea, and cellulose and its derivatives such as sodium carboxymethyl cellulose, ethyl cellulose and cellulose acetate; malt; gelatin; as well as other non-toxic compatible lubricants such as sodium lauryl sulfate and magnesium stearate, releasing agents, coating agents and other excipients suitable for use in manufacturing preparations, in solid, semisolid or liquid form and in addition auxiliary, stabilizing, thickening and coloring agents may be used. For preparing solid compositions such as tablets or capsules, the extract is mixed with a pharmaceutical excipient (e.g., conventional tableting ingredients such as corn starch, lactose, sucrose, sorbitol, talc, stearic acid, magnesium stearate, dicalcium phosphate or gums) and other pharmaceutical diluents (e.g., water) to form a solid composition. This solid composition is then subdivided into unit dosage forms containing an effective amount of the composition of the present invention. The tablets or pills containing the extract can be coated or otherwise compounded to provide a dosage form affording the advantage of prolonged action.

In an aspect, the present invention relates to a method for the treatment of a metabolic disorder comprising administering to a subject in need thereof a therapeutically effective amount of an extract of Momordica Charantia containing one or more nitrogen containing heterocyclic compounds selected from the group consisting of uridine, adenine and 2-hydroxy adenosine as the bioactive markers.

In an embodiment, the present invention relates to a method for the treatment of a metabolic disorder comprising administering to a subject in need thereof a therapeutically effective amount of an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres.

In an embodiment, the present invention relates to a method for the treatment of a metabolic disorder comprising administering to a subject in need thereof a therapeutically effective amount of an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres; and is substantially free from vicine.

In an embodiment, the present invention relates to a method for the treatment of a metabolic disorder comprising administering to a subject in need thereof a therapeutically effective amount of an extract of Momordica Charantia containing uridine, adenine and 2-hydroxy adenosine, as the bioactive markers.

In an embodiment, the present invention relates to a method for the treatment of a metabolic disorder comprising administering to a subject in need thereof a therapeutically effective amount of an extract of Momordica Charantia containing uridine, adenine and 2-hydroxy adenosine, as the bioactive markers: which is enriched with natural vitamin C and fibres.

In an embodiment, the present invention relates to a method for the treatment of a metabolic disorder comprising administering to a subject in need thereof a therapeutically effective amount of an extract of Momordica Charantia containing uridine, adenine and 2-hydroxy adenosine as the bioactive markers; wherein said extract is enriched with natural vitamin C and fibres: and is substantially free from vicine.

In an aspect, the present invention relates to a method for the treatment of a metabolic disorder comprising administering to a subject in need thereof a therapeutically effective amount of the composition recited above in one or more of the embodiments.

In one aspect, the present invention relates to a method for the treatment of a metabolic disorder comprising administering to a subject in need thereof a therapeutically effective amount of the composition comprising an extract of Momordica Charantia containing one or more nitrogen containing heterocyclic compounds selected from the group consisting of uridine, adenine and 2-hydroxy adenosine as the bioactive markers; wherein said extract is enriched with natural vitamin C and fibres; and is substantially free from vicine; and wherein the said composition either contains the extract alone or along with at least one pharmaceutically acceptable excipient.

In an embodiment, the present invention relates to a method for the treatment of a metabolic disorder comprising administering to a subject in need thereof a therapeutically effective amount of the composition comprising an extract of Momordica Charantia containing uridine, adenine and 2-hydroxy adenosine as the bioactive markers; wherein said extract is enriched with natural vitamin C and fibres; and is substantially free from vicine; and wherein the said composition either contains the extract alone or along with at least one pharmaceutically acceptable excipient.

According to an embodiment, the metabolic disorder is selected from: insulin resistance, hyperglycemia, diabetes (type 1 or type 2 diabetes), secondary complications associated with diabetes, obesity, glucose intolerance, hypercholesterolemia, dyslipidemia, hyperinsulinemia, atherosclerotic disease, polycystic ovary syndrome, coronary artery disease, metabolic syndrome, hypertension, or a related disorder associated with abnormal plasma lipoprotein, triglycerides or a disorder related to glucose levels such as pancreatic β cell regeneration.

According to an embodiment, the metabolic disorder is selected from insulin resistance, hyperglycemia, diabetes, secondary complications associated with diabetes, obesity, glucose intolerance, metabolic syndrome or a disorder related to glucose levels such as pancreatic β cell regeneration.

According to an embodiment, the metabolic disorder is diabetes or secondary complications associated with diabetes.

According to an embodiment, the metabolic disorder is diabetes which is type 2 diabetes.

According to an embodiment, the metabolic disorder is secondary complications associated with diabetes.

Accordingly, in an embodiment, the present invention relates to a method for the treatment of diabetes or secondary complications associated with diabetes comprising administering to a subject in need thereof; a therapeutically effective amount of an extract of Momordica Charantia containing one or more nitrogen containing heterocyclic compounds selected from uridine, adenine and 2-hydroxy adenosine as the bioactive markers; which is enriched with natural vitamin C and fibres; and is substantially free from vicine.

Accordingly, in an embodiment, the present invention relates to a method for the treatment of diabetes or secondary complications associated with diabetes comprising administering to a subject in need thereof; a therapeutically effective amount of an extract of Momordica Charantia containing uridine, adenine and 2-hydroxy adenosine as the bioactive markers; which is enriched with natural vitamin C and fibres; and is substantially free from vicine.

In an embodiment, the diabetes is type 2 diabetes.

Accordingly, in an embodiment, the present invention relates to a method for the treatment of type 2 diabetes comprising administering to a subject in need thereof a therapeutically effective amount of an extract of Momordica Charantia containing one or more nitrogen containing heterocyclic compounds selected from uridine, adenine and 2-hydroxy adenosine as the bioactive markers; which is enriched with natural vitamin C and fibres; and is substantially free from vicine.

Accordingly, in an embodiment, the present invention relates to a method for the treatment of type 2 diabetes comprising administering to a subject in need thereof a therapeutically effective amount of an extract of Momordica Charantia containing uridine, adenine and 2-hydroxy adenosine as the bioactive markers; which is enriched with natural vitamin C and fibres; and is substantially free from vicine.

Accordingly, in an embodiment, the present invention relates to a method for the treatment of secondary complications associated with diabetes comprising administering to a subject in need thereof a therapeutically effective amount of an extract of Momordica Charantia containing one or more nitrogen containing heterocyclic compounds selected from the group consisting of uridine, adenine and 2-hydroxy adenosine as the bioactive markers; which is enriched with natural vitamin C and fibres; and is substantially free from vicine.

Accordingly, in an embodiment, the present invention relates to a method for the treatment of secondary complications associated with diabetes comprising administering to a subject in need thereof a therapeutically effective amount of an extract of Momordica Charantia containing uridine, adenine and 2-hydroxy adenosine as the bioactive markers; which is enriched with natural vitamin C and fibres; and is substantially free from vicine.

In another embodiment, the secondary complications associated with diabetes are selected from the group consisting of diabetic neuropathy, diabetic cataract and retinopathy, diabetic corneal keratopathy, diabetic nephropathy, diabetic dermopathy, diabetic foot ulcers and other diabetic microangiopathies.

The selected dosage level depends upon a variety of factors including the activity of the particular extract of the present invention employed, the route of administration, the time of administration, the rate of excretion of the particular composition being employed, the duration of the treatment, used in combination with the other extracts, the age, sex, weight, condition, general health and prior medical history of the patient being treated, and like factors well known in the medical arts. In general, however, doses employed for adult human treatment will typically be in the range of 0.02-5000 mg per day or 1-1500 mg per day. The desired dose may conveniently be presented in a single dose or as divided doses administered at appropriate intervals, for example as two, three, four or more sub-doses per day.

It will be appreciated that compositions, medicaments and extracts according to the present invention can be used alone or alternatively can also be used in combination with other plant extracts, compositions or therapeutically active compounds (provided that those compounds do not inhibit the anti-diabetic properties of the extract or herbal composition according to the invention). Accordingly, the present invention also relates to the extract or the composition containing the extract as recited herein; for use in combination with a further ‘therapeutically active agent’ for the treatment of a metabolic disorder, particularly diabetes or complications associated with diabetes.

The therapeutically active agent can be selected from the known drugs or bioactive substances, including, but not limited to, orlistat, pioglitazone, rosiglitazone, glibenclamide, glipizide, glimeperide, repaglinide, nateglinide, or metformin.

In the aspects and embodiments of the present invention as well as the appended claims, it is intended to refer to the fruit of Momordica Charantia whenever reference to Momordica Charantia is made.

The following examples illustrate the invention. They do not however, limit the invention in any way. In this regard, it is important to understand that the particular assay used in the Examples section is designed only to provide an indication of anti-diabetic activity. There are many ways available to determine such activity, and a negative result in any one particular way is therefore not determinative.

The term once described, the same meaning applies for it, throughout the patent.

### EXAMPLE 1

### Preparation of the Extract of Momordica Charantia

### Step-I:

### Preparation of Amla Pulp

The fresh, amla fruits commercially available, were obtained from local market from Mumbai, Maharashtra, India were sorted out for any physical damage and cleaned to remove the superfluous particles from the fruits. The cleaned fruits were out into small pieces. The pieces of amla fruits were taken in a mixer along with equal amount of water (1:1) and the mixture was ground well in order to obtain a uniform juice of the fruit. The lumps or other parts of the fruits, if any, are removed from the juice. This obtained juice was directly used for the pH adjustment of the Momordica Charantia juice extract.

### Step-II

### Preparation of the Extract of Momordica Charantia:

### Step-IIA:

The fresh unripe green fruits of Momordica Charantia along with seeds commercially available, were obtained from local market from Mumbai, Maharashtra, India, were cleaned to remove the superfluous particles from the fruits. The cleaned fruits were cut into the small pieces and charged in a mill. The small pieces of fruit charged in the mill were ground during which the distilled demineralised water was added intermittently. The crude juice was obtained and the same was weighed.

### Step-IIB:

The crude juice obtained in step IIA was filtered using centrifuge, filter press, sieve or nylon cloth (100 mesh nylon cloth) to obtain clear juice extract with suspended particles. The residue that was left over after the filtration was removed. The filtrate (the clear juice extract) was taken for further treatments. The pH of the juice extract with the suspended particles was adjusted from 4 to 4.5 by the addition of Amla pulp (270 ml per litre of the juice extract) prepared as per the procedure given in step I, with continuous stirring. The stirring of juice was continued for 10-15 minutes after adding the Amla pulp to obtain the juice extract having pH in the aforesaid range. The pH of the juice extract is checked after stirring and if the pH of the juice extract is not in the specified range, it is required to add Amla pulp. The juice extract was allowed to stand for 5 to 25 minutes.

### Step-IIC

The stabilised juice extract obtained in step IIB above was neutralized to a pH range of 7 to 7.2 by adding 15% Sodium hydroxide (NaOH) solution (10 mL/litre of the juice extract). The neutralised juice extract was stirred continuously for 10-15 minutes after the addition of NaOH solution. The pH of the juice extract is checked to ensure that it is in the specified range, if not, NaOH solution is further added. This is followed by addition of 0.15% (15 g/litre of juice extract) of microcrystalline cellulose and 0.15% (15 g/litre of juice extract) of Aerosil to the neutralized juice extract and the mixture was stirred well for 10 minutes to get the homogeneous mixture of juice extract.

### Step-IID

The homogenous juice extract, obtained in step IIC, was transferred to a chamber and was concentrated to semi solid consistency mass by means of distillation at the temperature of 60° C.±5° C. at reduced pressure [700 mm of Hg]. The concentrated juice was subjected to drying using a suitable dryer such as freeze dryer or spray dryer at 55 ° C.±5° C. for complete removal of the water to obtain juice extract powder.

The yield of the extract calculated was 4.0-4.5 Kg with respect to fresh fruits of Momordica Charantia. The ratio of fresh fruits Momordica Charantia to the extract was found to be 25:1.

Extract so obtained in Step IID is referred to herein as “Extract of Example 1”.

### EXAMPLE 2

### Preparation of Capsules

The Extract of Example 1 and the excipients (microcrystalline cellulose, aerosol, Dicalcium phosphate) were sifted into the mass mixer through 30 mesh and 40 mesh respectively into the mass mixer and the sifted materials were mixed properly. The blend was then granulated using Povidone (PVPK-30) in order to obtain the wet granules. The wet granules were dried into the tray dryer at the temperature of 50° C.±5° C. The dried granules were sifted into the mass mixer. The lubricant materials (Talc and Magnesium stearate) were sifted and mixed properly with dried granules into mass mixer. The obtained granules were filled into hard gelatine capsule shells. The composition of each capsule containing Extract of Example 1 is given below.

| Ingredient | Quantity in mg/capsule |
|---|---|
| Extract of Example 1 | 400 |
| Microcrystalline Cellulose | 34.97 |
| Colloidal Silicon Dioxide | 8.55 |
| Dicalcium Phosphate | 70.00 |
| Povidone (PVPK-30) | 11.4 |
| Sodium Benzoate | 2.85 |
| Potassium sorbate | 1.14 |
| Sodium Starch Glycolate | 17.10 |
| Croscarmellose sodium | 17.10 |
| Purified Talcum | 5.70 |
| Magnesium Stearate | 2.85 |

### EXAMPLE 3

### Stability Studies of Capsules

### 3A: Method for Determining Content of Bitter:

3 g of sample and 0.5 g calcium carbonate was added in a 250 mL beaker. Pumice pieces were added, the mixture was then extracted with boiling distilled water to obtain powder. The powder obtained was allowed to settle and the supernatant liquid was filtered through cotton in another flask. The extraction was repeated to obtain pale yellow extract. The extracted liquid was collected in a 250 mL beaker, boiled and concentrated to obtain 10 mL of concentrated extract. Rectified spirit (alcohol) 20 mL was then added and kept on a water bath. As it starts boiling it was removed, the residue was allowed to settle and the supernatant liquid was filtered through filter paper. The extraction was continued to obtain pale yellow extract. The pale yellow extract obtained was evaporated in evaporating dish and kept on water bath to evaporate alcohol completely. The residue obtained was diluted with about 15 mL distilled water, transferred in a separator and extracted with ethyl acetate (4-5 times) to complete the extraction. Ethyl acetate extract obtained was given washing with distilled water, the water layer was discarded and ethyl acetate layer was evaporated on water bath to obtain the residue. The residue obtained was kept in an oven at 105° C., cooled it in a desiccator to attain room temperature and was weighed to obtain the constant weight.

The bitter content was determined according to the following formula:

Bitter (% w/w)=Weight of the residue×100/Weight of the sample

Bitter (mg/capsule)=Bitter (% w/w)×(average filled weight in g×1000/100)

### 3B: Method for Determining Content of Proteins (by Automated Protein Analyzer):

Digestion: 1 g of sample was taken in a Kjeldahl tube. Sodium sulphate (9.95 g), copper sulphate (0.05 g) and concentrated sulphuric acid (20 mL) were added in the tube. Samples were digested for 30 minutes after a clear.

Distillation: Kjeldahl tube was fitted to the distillation unit. Water 10 mL and 32% sodium hydroxide (90 mL) was added to the sample. Distillation was carried out and the distillate was collected in 60 mL of 4% Boric acid solution of pH 4.65.

Titration: Boric acid solution was titrated with 0.25 M (0.5N) sulphuric acid to pH 4.65. Blank and sample were titrated.

The protein content was determined according to the following formula:

Calculations:

$$\% proteins ( w \text{/} w ) = \frac{( Sample reading - Blank reading ) \times 1.4007 \times 6.25 \times NF}{Sample weight}$$

wherein,

NF represents Normality Factor of 0.5 N sulphuric acid;

1.4007 represents a single factor that takes into account the molecular weight of nitrogen, the conversion of the milliequivalent result of V*N, and the conversion to % 6.25 represents protein factor.

Protein (mg/capsule)=Protein (% w/w)×(average filled weight in g×1000/100)

3C: Microbiological tests: The Microbiological tests were performed as per United States Pharmacopoeia-37, volume 1, 2014, chapters 2021, 2022 and 2023 specification and British Pharmacopoeia volume V, 2014, Appendix XVI F and G (A474-A476).

TABLE 1
Stability studies of capsules

| Parameters | Initial | | 1 Month | | 2 Months | | 3 Months | | 6 Months | |
|---|---|---|---|---|---|---|---|---|---|---|
| Average weight | 630.03 | mg | 640.05 | mg | 622.75 | mg | 630.16 | mg | 632.96 | mg |
| of Capsule | | | | | | | | | | |
| Average net filled | 515.24 | mg | 522.90 | mg | 507.60 | mg | 521.55 | mg | 515.90 | mg |
| weight of capsule | | | | | | | | | | |
| Loss on drying | 10.531% | | 12.493% | | 10.431% | | 11.908% | | 9.294% | |
| Content of Bitter | 11.690 | mg/capsule | 10.055 | mg/capsule | 10.365 | mg/capsule | 14.100 | mg/capsule | 12.108 | mg/capsule |
| Content of | 74.24 | mg/capsule | 70.591 | mg/capsule | 70.75 | mg/capsule | 69.85 | mg/capsule | 63.713 | mg/capsule |
| Proteins | | | | | | | | | | |
| Estimation of | 0.89 | mg/capsule | 0.89 | mg/capsule | 0.88 | mg/capsule | 0.92 | mg/capsule | 0.92 | mg/capsule |
| Uridine by HPLC | | | | | | | | | | |
| Total aerobic | 90 | cfu/g | 90 | cfu/g | 80 | cfu/g | 60 | cfu/g | 180 | cfu/g |
| microbial count | | | | | | | | | | |
| Total yeast, | <10 | cfu/g | <10 | cfu/g | <10 | cfu/g | <10 | cfu/g | <10 | cfu/g |
| mould, fungi | | | | | | | | | | |
| count | | | | | | | | | | |
| Bile tolerant | <10 | cfu/g | <10 | cfu/g | <10 | cfu/g | <10 | cfu/g | <10 | cfu/g |
| gram negative | | | | | | | | | | |
| bacteria | | | | | | | | | | |

| | | | | | |
|---|---|---|---|---|---|
| Escherichia coli | Absent | Absent | Absent | Absent | Absent |
| Salmonella spp. | Absent | Absent | Absent | Absent | Absent |
| Staphylococcus | Absent | Absent | Absent | Absent | Absent |
| aureus | | | | | |
| Pseudomonas | Absent | Absent | Absent | Absent | Absent |
| aeruginosa | | | | | |
| Clostridium spp. | Absent | Absent | Absent | Absent | Absent |

Conclusion: It was observed that the capsules packed in HDPE container are stable at 40° C. over a period of 6 months.

### EXAMPLE 4

### Determination of Content of Bioactive Markers in the Extract of Example 1

The content of bioactive markers (uridine, adenine and 2-hydroxy adenosine) present in the Extract of Example 1 of Momordica Charantia was determined by the chromatographic method such as the HPLC method. It was found that the extract of example 1 contains uridine (0.3%), adenine (0.2%) and 2-hydroxyadenosine (0.3%).

### Method

A. Uridine Standard solution: 5.0 mg of Uridine was transferred into a 50 mL volumetric flask, 25 mL of diluent (Water: Methanol 90:10) was added and was sonicated. 5 ml of solution was pipette out and transferred into a 25 ml volumetric flask.

B. Sample Preparation: Dissolved 0.5 g of Extract of Example 1 in 25 mL of water:methanol (90:10). The mixture was filtered through whattmann filter paper No. 1 and filtrate analyzed by HPLC.

### Analytical HPLC Conditions:

Column: Hypersil BDS C-18, 250 mm×4.6 mm, 5 μ
Mobile phase A: Phosphate Buffer 3.0
Mobile phase B: acetonitrile
Gradient: time (minutes)/% A: 0/90, 25/60, 30/20,35/20,36/90,40/90
Flow rate: 1.0 mL/minute.
Detector: UV
Detection wavelength: 261 nm.
Injection volume: 20 μL
Run time: 40 minutes
Temperature: Ambient
Diluent: water:methanol (90:10)

The content of Uridine was determined according to the following formula:

### Calculation:

Content of Uridine (% w/w)=(Sample Area/Standard Area)×(Dilution factor of Standard/Dilution factor of Sample) X % Purity of Standard

Uridine (mg/capsule)=Uridine (% w/w)×(average filled weight in g×1000/100)

### EXAMPLE 5

### Determination of the Content of Vicine in the Extract of Example 1

The commercially available juice extracts of Momordica Charantia suffer from drawbacks due to the presence of neurotoxins which can adversely affect function in both developing and mature nervous tissue during the prolonged administration. Vicine is one of the widely known neurotoxins, which is toxic and capable of causing severe adverse effects.

The extract of Momordica Charantia of the present invention was analysed to determine content of vicine, if any, by using the chromatographic method and based on the analysis it was found that Extract of Example 1, is free from vicine. Comparative data in respect of the content of vicine in the Extract of Example 1 Momordica Charantia with the commercially available extracts of Momordica Charantia is presented in Table-2. The commercially available extracts are referred to herein as Reference Extract 1, Reference Extract 2, Reference Extract 3, Reference Extract 4, Reference Extract 5, Reference Extract 6, Reference Extract 7, Reference Extract 8, and Reference Extract 9.

TABLE 2
Comparison of the content of vicine in the extract with the commercially
available juice extracts of Momordica Charantia

| | Extracts of | | |
|---|---|---|---|
| | Momordica | | Vicine content |
| Sr. No | Charantia | Batch No | (%) |
| 1 | Reference Extract 1 | KP/MCF/001/12 | 0.512 |
| 2 | Reference Extract 2 | MC0313001 | 0.187 |
| 3 | Reference Extract 3 | TAMC1214 | 0.109 |
| 4 | Reference Extract 4 | KP/MC/013/12 | 0.671 |
| 5 | Reference Extract 5 | KP/MC/014/12 | 0.065 |
| 6 | Reference Extract 6 | KKL/746 | 0.259 |
| 7 | Reference Extract 7 | 165033 | 3.761 |
| 8 | Reference Extract 8 | KP/05052013 | 0.455 |
| 9 | Reference Extract 9 | CL/MC/001/13 | 0.963 |
| 10 | Extract of Example 1 | 019022013 | Not detected |

### Biological Activity

### EXAMPLE 6

A patient having non-insulin dependent diabetes mellitus (Type 2 diabetes) is administered with three capsules of the Extract of Example 1 per day. Each capsule contains 400 mg of the extract of example 1 as per the Example 2

The Extract of Example 1 is found to have an activity on lowering the blood glucose level of the patient undergoing the treatment.

1. An extract of Momordica Charantia containing one or more nitrogen containing heterocyclic compounds selected from the group consisting of uridine, adenine and 2-hydroxy adenosine as the bioactive markers.

2. The extract according to claim 1, wherein the said extract contains uridine, adenine and 2-hydroxy adenosine as the bioactive markers.

3. The extract according to claim 1, wherein the said extract contains about 0.1% to about 5% by total weight of uridine, adenine and 2-hydroxy adenosine as the bioactive markers.

4. The extract according to claim 1, wherein the said extract enriched with natural vitamin C and fibres, and wherein the said extract is substantially free from vicine.

5. A composition comprising a therapeutically effective amount of a standardised extract of Momordica Charantia containing one or more of nitrogen containing heterocyclic compounds selected from the group consisting of uridine, adenine and 2-hydroxy adenosine as the bioactive markers, either alone or along with at least one pharmaceutically acceptable excipient.

6. The composition according to claim 5, wherein the said standardised extract contains uridine, adenine and 2-hydroxy adenosine as the bioactive markers.

7. The composition according to claim 5, wherein the said standardised extract contains about 0.1% to about 5% by total weight uridine, adenine and 2-hydroxy adenosine as the bioactive markers.

8. The composition according to claim 5, wherein the said standardised extract is enriched with natural vitamin C and fibres, and wherein the said extract is substantially free from vicine.

9. The composition according to claim 5, wherein the said composition is provided for oral administration.

10. A method for the treatment of a metabolic disorder comprising administering to a subject in need thereof a therapeutically effective amount of the extract as claimed in claim 1.

11. A method for the treatment of a metabolic disorder comprising administering to a subject in need thereof a therapeutically effective amount of the composition as claimed in claim 5.

12. The method according to claim 10, wherein the metabolic disorder is diabetes or secondary complications associated with diabetes.

13. The method according to claim 12, wherein the metabolic disorder is diabetes.

14. The method according to claim 12, wherein the diabetes is type 2 diabetes.

15. The method according to claim 12, wherein the metabolic disorder is secondary complications associated with diabetes.

16. A process for the preparation of a standardised extract of Momordica Charantia comprising the steps of:
a) preparing juice from fresh unripe green fruits of Momordica Charantia along with seeds;
b) filtering the juice as obtained in step (a) to obtain an extract with suspended particles;
c) altering pH of the juice extract as obtained in step (b) to the acidic pH range by the addition of a natural tonic;
d) allowing the juice extract as obtained in step (c) to stand;
e) neutralising the pH of the juice extract as obtained in step (d) by using a base or alkali;
f) adding excipients to the juice extract as obtained in step (e) to obtain a homogenous mixture;
g) concentrating the juice extract as obtained in step (f) to a semi solid mass under distillation;
h) drying the concentrated juice extract as obtained in step (g) to obtain dried extract of Momordica Charantia; and
i) determining the amount of bioactive markers in the extract of Momordica Charantia; wherein the bioactive markers are nitrogen containing heterocyclic compounds selected from the group consisting of uridine, adenine and 2-hydroxy adenosine.
